(19) United States
(12) Patent Application Publication
(10) Pub. No.: US 2022/0226141 A1
(43) Pub. Date: Jul. 21, 2022

(54) SUPPORT APPARATUS

(71) Applicant: Peter Kioko, Stafford, VA (US)

(72) Inventor: Peter Kioko, Stafford, VA (US)

(21) Appl. No.: 17/577,126

(22) Filed: Jan. 17, 2022

Related U.S. Application Data
(60) Provisional application No. 63/139,650, filed on Jan. 20, 2021.

(51) Int. Cl. A61F 5/058

(52) U.S. Cl. CPC A61F 5/05891

(57) ABSTRACT

The disclosure comprises a support apparatus comprising a body. The body comprises an adjustment member and a chin support member. The adjustment member and the chin support member are coupled together. The adjustment member includes a frame. The frame comprises a bendable region. The bendable region also comprises a plurality of alignment tubes that are coupled to a stability sheet. The adjustment member and the chin support member comprise padding and a cover. The apparatus also includes an adjustment strap coupled to the adjustment member and is configured to hold the adjustment member at a desired angle. The apparatus also includes a support strap. The support strap is configured to wrap around the torso of the user to stabilize the adjustment member and chin support member to the user's shoulder area.

### RELATED APPLICATIONS

Under provisions of 35 U.S.C. § 119(e), the Applicant claim the benefit of U.S. provisional application No. 63/139,650, filed Jan. 20, 2021, which is incorporated herein by reference.

It is intended that each of the referenced applications may be applicable to the concepts and embodiments disclosed herein, even if such concepts and embodiments are disclosed in the referenced applications with different limitations and configurations and described using different examples and terminology.

### TECHNICAL FIELD

The present disclosure relates generally to a support apparatus, and more particularly, to a system a head support system.

### BACKGROUND OF THE INVENTION

When a person places their body in different positions, additional stresses on joints may cause the person to feel discomfort or pain. These positions can range from standing, sitting, and even laying in a prone position. A person may need additional support to provide additional relief. When the person is also required to move, the amount of initial pain can be increased. Most current forms of support do not provide the necessary stability when a person seeks to both move and/or remain stationary. Thus, there exists a need for a solution that addresses head support with stability reinforcement.

### SUMMARY OF THE INVENTION

A support apparatus may be provided. This brief overview is provided to introduce a selection of concepts in a simplified form that are further described below in the Detailed Description. This brief overview is not intended to identify key features or essential features of the claimed subject matter. Nor is this brief overview intended to be used to limit the claimed subject matter's scope.

The present disclosure meets the needs described above comprising a system for a head support. An embodiment of the disclosure can comprise a support apparatus. The support apparatus can be a body. The body comprises an adjustment member and a chin support member. The adjustment member and the chin support member can be coupled together. The adjustment member includes a frame, and the frame comprises a bendable region. The bendable region also comprises a plurality of alignment tubes and a stability sheet, wherein an exterior surface of the plurality of alignment tubes are coupled to the stability sheet. The support apparatus also includes a support strap that wraps around the user's torso and couples to the body of the support apparatus to restrict movement of the support apparatus.

Another embodiment of the disclosure comprises a method for using the support apparatus. The method can initially comprise providing a support apparatus. The support apparatus can include a body. The body can comprise an adjustment member including a frame. The frame comprises a bendable region, such that the bendable region includes a stability sheet and a plurality of alignment tubes. The stability sheet is coupled to an exterior surface of at least one of the plurality of alignment tubes. The support apparatus also includes and a support strap detachably coupled to the body. The support strap can also include at least one support loop. The method of using the support apparatus can comprise engaging the support apparatus in proximity to the head of a user by place the support apparatus near the shoulder area of the user. The method can include adjusting the frame of the adjustment member. Adjusting the frame can include causing the frame to bend from a prone position to an angled position. The method can include engaging the adjustment member to a surface of the user' head. The method can also include coupling a support strap to the apparatus. The support strap can also be engaged to the torso of the user by wrapping the strap around the torso of the user.

In view of the foregoing, it will be appreciated that the present disclosure provides a system for a head support. The specific systems and techniques for accomplishing the advantages described above will become apparent from the following detailed description of the embodiments and the appended drawings and claims.

Both the foregoing brief overview and the following detailed description provide examples and are explanatory only. Accordingly, the foregoing brief overview and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

FIG. 1 depicts an isometric top view of the support apparatus consistent with the present disclosure.

FIG. 2 depicts a front view of the support apparatus in use consistent with the present disclosure.

FIG. 3 depicts a front view of an alternative embodiment of the support apparatus in use consistent with the present disclosure.

FIG. 4 depicts an isometric partial view of the support apparatus consistent with the present disclosure.

FIG. 5 depicts a cross-sectional view of the support apparatus as depicted in FIG. 4 consistent with the present disclosure.

FIG. 6 A-E depicts a partial side view of alternative embodiments of the frame consistent with the present disclosure.

FIG. 7 depicts a side view of the support strap.

FIG. 8 depicts a rear view of the support apparatus in use consistent with the present disclosure

FIG. 9 depicts a front view of an alternative embodiment of the support apparatus in use, without the detachable chin support member, consistent with the present disclosure.

FIG. 10 depicts a flowchart of the method for using the present disclosure.

### DETAILED DESCRIPTION

Illustrative embodiments of the disclosure will now be described more fully hereinafter with reference to the accompanying drawings, in which some, but not all embodiments of the disclosure are shown. The disclosure may be embodied in many different forms and should not be construed as limited to the embodiments set forth herein; rather, these embodiments are provided so that this disclosure will satisfy applicable legal requirements.

Whenever appropriate, terms used in the singular also will include the plural and vice versa. The use of “a” herein means “one or more” unless stated otherwise or where the use of “one or more” is clearly inappropriate. The use of “or” means “and/or” unless stated otherwise. The use of “comprise,” “comprises,” “comprising,” “include,” “includes,” and “including” are interchangeable and not intended to be limiting. The term “such as” also is not intended to be limiting. For example, the term “including” shall mean “including, but not limited to.”

The following description is provided as an enabling teaching of the disclosed articles, systems, and methods in their best, currently known embodiments. To this end, those skilled in the relevant art will recognize and appreciate that many changes can be made to the various aspects of the articles, systems, and methods described herein, while still obtaining the beneficial results of the disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

As used throughout, the singular forms “a,” “an” and “the” include plural referents unless the context clearly dictates otherwise. Thus, for example, reference to “a gasket” can include two or more such gaskets unless the context indicates otherwise.

As used throughout, “substantially” with respect to a measure can refer to a range of values comprising +/−10 degrees. For example, substantially orthogonal, normal, or parallel can include embodiments, where the referenced components are oriented +/−10 degrees of being classified as orthogonal, normal, or parallel respectively.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

The word “or” as used herein means any one member of a particular list and also includes any combination of members of that list.

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present disclosure contains headers. It should be understood that these headers are used as references and are not to be construed as limiting upon the subjected matter disclosed under the header.

The present disclosure includes many aspects and features. Moreover, while many aspects and features relate to, and are described in, the context of support apparatus 100, embodiments of the present disclosure are not limited to use only in this context.

Consistent with embodiments of the present disclosure, a support apparatus may be provided. This overview is provided to introduce a selection of concepts in a simplified form that are further described below. This overview is not intended to identify key features or essential features of the claimed subject matter. Nor is this overview intended to be used to limit the claimed subject matter's scope.

Both the foregoing overview and the following detailed description provide examples and are explanatory only. Accordingly, the foregoing overview and the following detailed description should not be considered to be restrictive. Further, features or variations may be provided in addition to those set forth herein. For example, embodiments may be directed to various feature combinations and sub-combinations described in the detailed description.

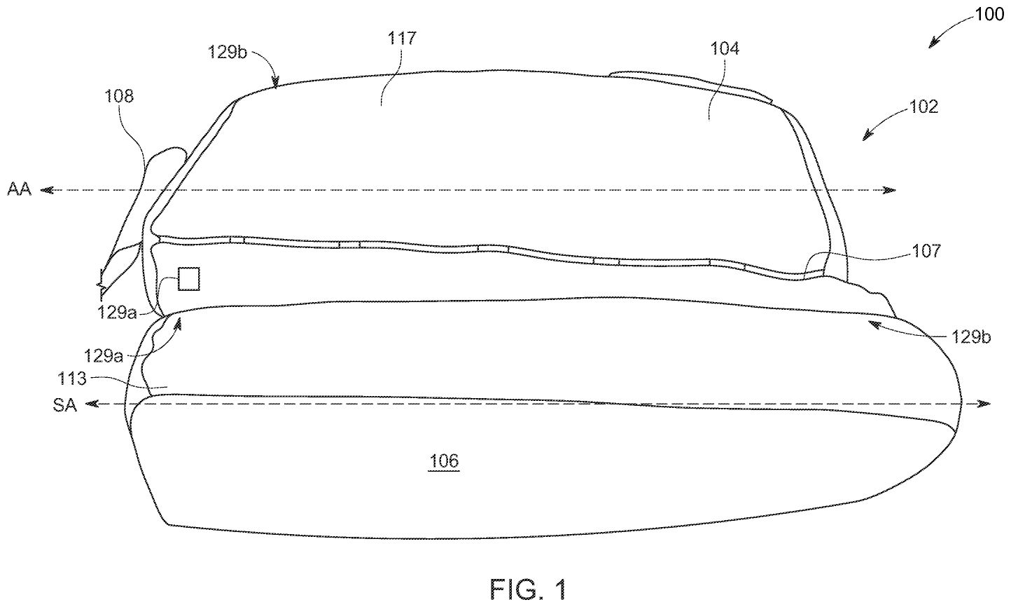

As shown in FIG. 1, the support apparatus 100 can comprise a body 102. The body 102 can comprise an adjustment member 104 and a chin support member 106. The adjustment member 104 and the chin support member 106 can both comprise members with respective lengths and can diametrically opposed ends. In a further aspect, the adjustment member 104 and the chin support member 106 can be oriented along an adjustment member axis (AA) and a chin support member axis (SA), respectively. The adjustment member axis (AA) and the chin support member axis (SA) can be oriented parallel to each other, when the support apparatus is not in use. In another aspect, the adjustment member 104 and the chin support member 106 can be coupled together at a respective end of each section. For example, the adjustment member 104 and the chin support member 106 can be coupled together with alignment fasteners 129A and 129B, such as hook and loop connectors, buttons, snaps. The alignment fasteners 129A-B located on the ends of the adjustment member 104 and chin support member 106 provide additional usage flexibility for user of the apparatus 100. If the user needs support on the right side of their head, first set of alignment fasteners 129A on the chin support member 106 and the adjustment member 104 can be coupled. If the user needs support on left side of their head, the adjustment member 104 can be rotated and the second set of alignment fasteners 129B on the chin support member 106 and the alignment fastener 129B on the adjustment member 104 can be coupled.

The body 102 can be encompassed with a cover material 113. In one aspect, the material can a woven and/or plush fabric. In one or more aspects, the cover material 113 of the body 102 may further comprise at least one of: a slip-resistant material, a foam, a rubber, a Polyvinyl Chloride (PVC), and other known materials. In a further aspect, the cover material 113 can comprise wicking properties. The wicking properties can allow moisture to be drawn away from a surface in contact with the cover material 113, instead of trapping moisture between the cover material and the user's head. In another aspect, the cover material 113 can comprise a water-resistant material, such as rubber or blended fabric.

The adjustment member 104 and the chin support member of the body 102 can also comprise padding 117. In one or more aspects, the padding 117 of the body 102 comprise at least one of: a cushion, a cushioned support, filler, stuffing, gel contents, air bubbles, foam components, memory foam. In at least one aspect, the padding 117 can compression resilient. For example, the padding 117 can have an elevated indentation load deflection (ILD) rating wherein ILD is a unit of measurement which refers to the number of pounds of pressure or force that is needed to indent foam to a depth of four inches by twenty-five percent based on tests performed on an area of fifty square inches. In at least one aspect, the padding 117 of the body 102 may further comprise at least combinations of materials including: one of a viscoelastic foam, viscoelastic polyurethane foam, a low-resilience polyurethane foam, and a gel infused memory foam. Alternatively, the padding 117 of the body 102 may further comprise one or more layers of foam further comprising at least one of a viscoelastic foam, a hard firmness foam, a medium firmness foam, a soft firmness foam, a soft to medium lightweight viscoelastic layer of foam, or a gel like foam.

In some embodiments, the padding 117 can be configured to be at least one of air permeable, partially-permeable, and semi-permeable as desired for use. In one or more aspects, the padding 117 may further comprise can further comprise at least one external water-resistant layer that is configured to provide a waterproofing effect to the core and the plurality of impact absorbing layers. In another aspect, the padding can comprise a wicking layer similar to the cover material 113. The at least one water-resistant layer can comprise at least one of a waterproof coating, a rubberized coating, a room temperature vulcanization silicone, and a flexible polyurethane adhesive.

In another aspect, the adjustment member 104 and the chin support member 106 can define a head channel 107. The head channel 107 can serve as a passage to allow the user to comfortably place their head between the adjustment member 104 and the chin support member 106. The adjustment member 104 can also comprise an adjustment strap 108. The adjustment strap 108 can be a strand of material that aids the adjustment member 104 in maintaining the orientation angle (not shown) of the adjustment axis AA. Further, the adjustment strap 108 can comprise an adjustment strap fastener 115 that couples the adjustment strap 108 between two regions of the adjustment member 104. For example, the adjustment strap 104 can be oriented to span the ends of the adjustment member 104 and be configured to hold the adjustment member 104 in an angled (bent) configuration. The adjustment member fastener 115 can comprise any suitable device used to connect two ends of a length of material. For example, the fastener can comprise buttons, hook and loop, a buckle, or the like.

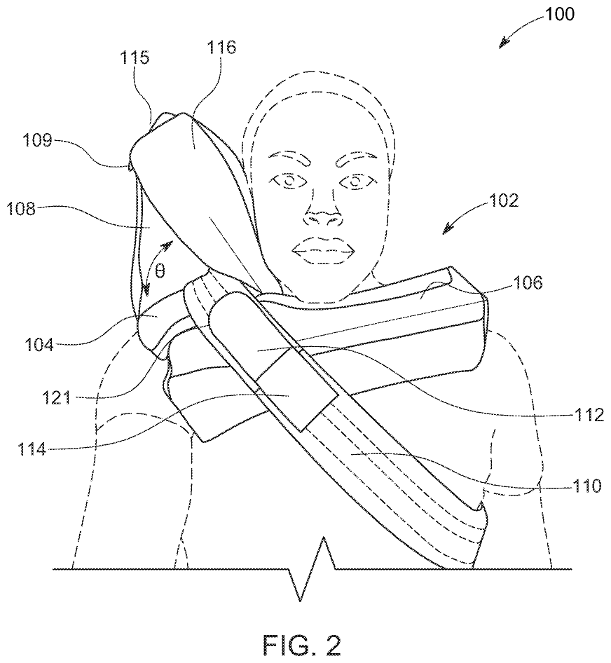

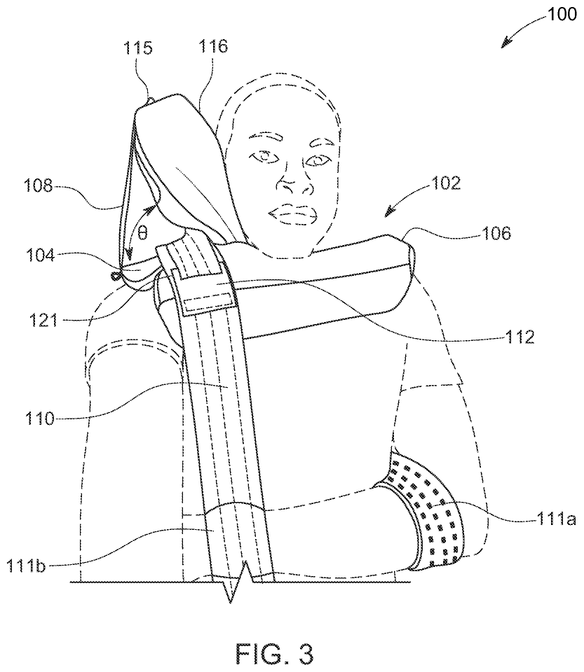

As shown in FIG. 2 and FIG. 3, the support apparatus 100 can further comprise a support strap 110. Similar to the adjustment strap 108, the support strap 110 can be comprised of a strand of woven fabric. In a further aspect, the support strap 110 can comprise support padding 133, similar to the various embodiments of the padding 117 of the body 102. The material comprising the adjustment strap 108 or support strap 110 can be braided or structurally oriented to endure tensile forces placed on these straps. The support strap 110 can be configured to engage the support apparatus 100 to the user's body. For example, the support strap 110 can wrap around the user's body. As shown in FIG. 3, the support strap 110 can wrap around the shoulder and under the arm of the user, similar to a sash. As shown in FIG. 3, the support strap 110 can have a longer length to facilitating engaging the user's shoulder and also wrapping around the arm and forearm of the user. The support strap 110 can also be detachable from the body 102.

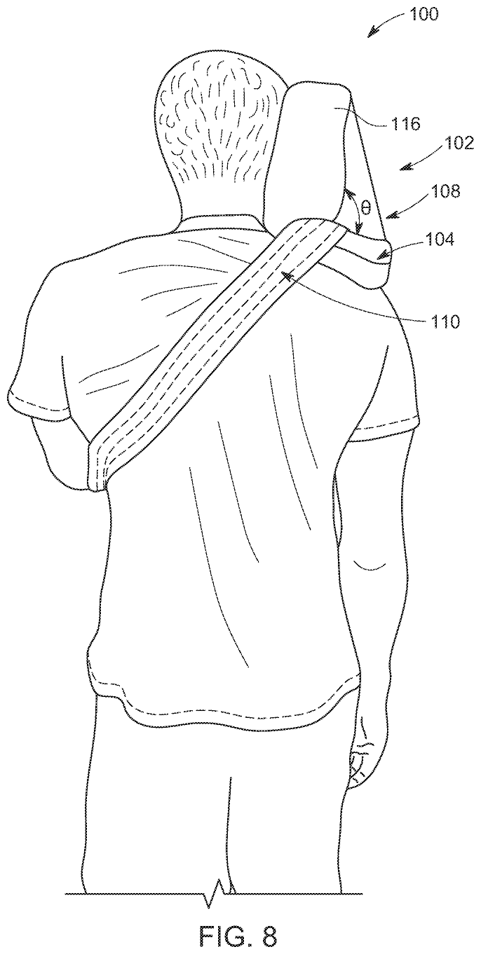

In one or more embodiments, the support strap 110 may be attached in various positions according to one or more recommended methods. In at least one embodiment, as depicted in FIG. 2, the support strap 110 may be positioned to wrap around the torso diagonally similar to a sash. In this configuration, the support strap 110 can be secured by wrapping around the adjustment member 104 and the opposite side of the torso underneath the arm. As shown in FIG. 8, the support strap 110 can wrap diagonally around the user's back. The support strap 110 can further comprise a support strap fastener 121 to restrict the motion of the support strap. For example, the fastener 121, can use Velcro® or similar operating fastener to couple opposing sides of the support strap 110.

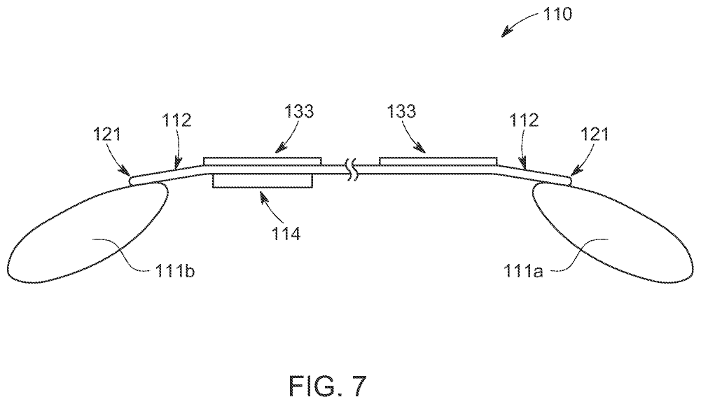

As shown in FIG. 7, the support strap 110 can comprise an elastic region 112. The elastic region 112 allows the support strap 110 to elongate to a length beyond an un-stretched length. The elastic region can comprise a stretchable material, such as spandex, spandex blend, rubber or the like. Similar to the adjustment strap 108, the fastener 121 can comprise any suitable embodiment used to connect two portions of the support strap 110. For example, the fastener can comprise buttons, hook and loop, a buckle, or the like. In yet a further aspect, the support strap 110 can also comprise a strap pocket 114. The strap pocket 114 can comprise a container with a lid that is configured to hold items for the user. For example, the strap pocket 114 can hold the user's cell phone, keys, or other personal items that the user desires quick access.

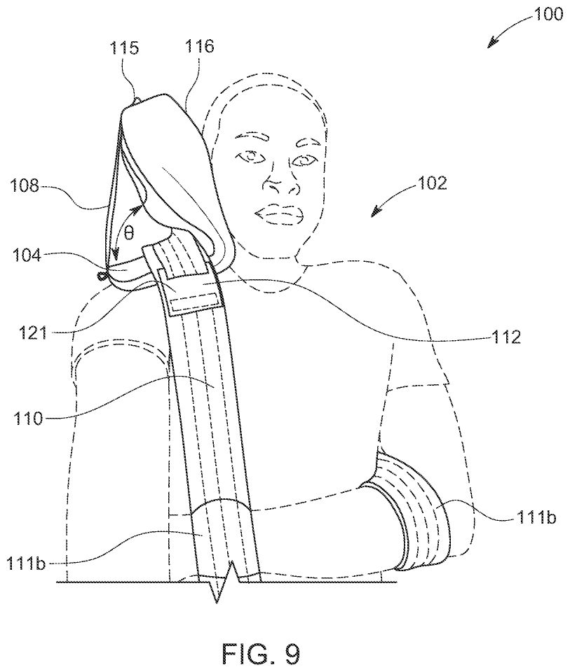

As shown in usage configurations in FIG. 3 and FIG. 9, the support strap 110 can comprise support loops 111A and 111B. The support loops 111A-B can comprise similar materials as the support strap 110. The support loops 111A-B provide an alternate wearing configuration for the user. After the user has adjusted, their head positioning in adjustment member 104. The user can wrap the support strap 110 over the top of the adjustment member 104 and around the user's torso, as shown in FIG. 8. The support loop 111B located at the end of the support strap 110, that is wrapped around the back of the torso, can be coupled to the user's forearm by passing their forearm through support loop 11B. The support loop 111A located at the opposing in of the support strap 110 can be coupled to the users' wrist, by passing the user's wrist through the loop. When the user allows the arm, that is engaged with the support loops 111A-B, to rest, the weight of the arm can hold the support apparatus 100 in place. An alternate embodiment can comprise an apparatus without a chin support member 106. As shown in FIG. 9, the user has the ability to detach 106 from 104 so that the body can be used without the chin support of 106 if the user so chooses.

Referring back to the adjustment member, the adjustment member 104 can also comprise a bendable region 116. The bendable region 116 can serve as the engagement point between the body 102 and the side of the user's head. The bendable region 116 can be adjusted such that an angle of orientation theta (0) is created between the adjustment axis AA and the bendable region 116. As mentioned earlier, the adjustment strap 108 can be used to hold bendable region 116 in a stationary position. In a further aspect, the adjustment strap 108 can also comprise an adjustment strap elastic region 109. The elastic regions for both the adjustment strap 108 and support strap 110 can comprise materials configured to elongate the strap when these straps are stretched or pulled. For example, the elastic bands can comprise elastic bands comprising arrangements of rubber or similar material.

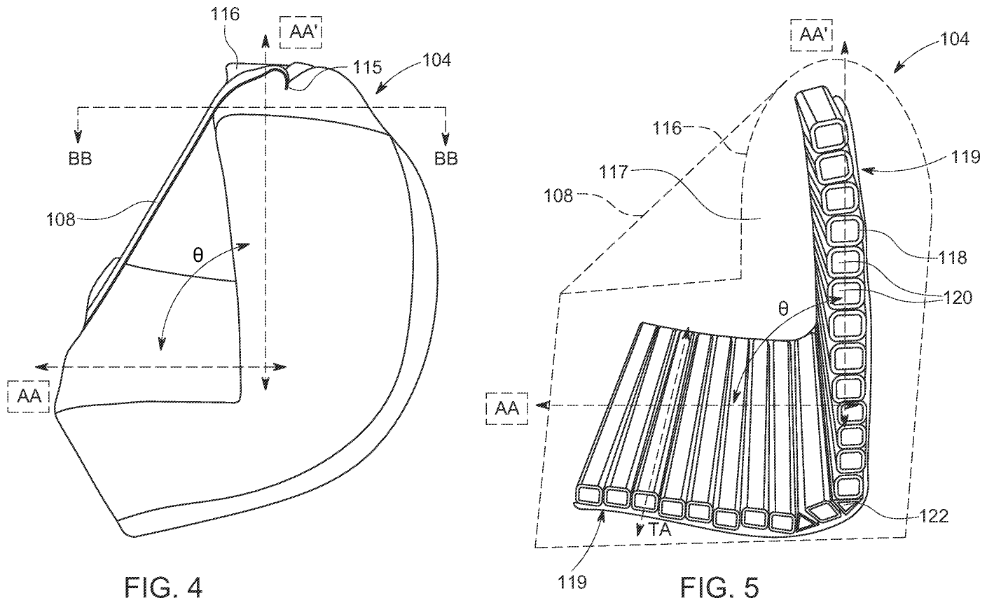

As shown in FIG. 4 and FIG. 5, the bendable region 116 of adjustment member 104 can be configured to pivot to an orientation angle θ. The bendable region 116 can be rotated to an orientation angle to provide additional support and comfort to the user's head. FIG. 5 is a cross-sectional view of FIG. 4 removing exterior cover material 113 and the padding 117. In a further aspect, the bendable region 116 can comprise a frame 118. The frame 118 can be a structure that provides modular support to the flexion of the bendable region 116. In one aspect, the frame 118 can comprise an arrangement of a plurality of alignment tubes 120 and a stability sheet 119. The alignment tubes 120 can be oriented so that they are parallel to each other. For example, each tube length axis (TA) can be substantially parallel to each other. As shown in FIG. 5, the length axis (TA) can be substantially perpendicular to the adjustment member axis AA. When a force is applied to the bendable region 116, while in the prone position, the bendable region 116 can then rotate around the vertex of orientation angle θ. The orientation angle θ is the angle between the adjustment member alignment axis AA and a secondary alignment axis AA′. The secondary axis AA′ is the alignment axis of the bendable region 116 that rotates from the prone position (being parallel to the adjustment member alignment axis AA) to an upright position (being substantially perpendicular to the adjustment member alignment axis AA). It is further considered, in an alternate embodiment that, that the frame 118 can also be structured wherein the tube length axis (TA) is substantially parallel to the adjustment member axis AA.

The alignment tubes 120 can be annular such that they are hollow along the length of the tube length axis (TA). The alignment tubes 120 can be hollowed out to reduce weight and cost while maintaining durability. In another aspect, the alignment tubes 120 can have a solid cross-section. The alignment tubes 120 can be solid while still reducing the overall weight and cost of manufacturing the support apparatus 102. In another aspect, the alignment tubes 120 can have a square cross-sectional shape. In a further aspect, the square cross-sectional shape can be rounded or chamfered at the external corners to allow two alignment tubes 120 to move relative to each other. In a further aspect, the frame 118 can further comprise a bending fulcrum 122. The bending fulcrum 122 can be a tube or member that allows adjacent alignment tubes 120 to bend around the bending fulcrum. As shown in FIG. 5, the fulcrum 122 cross-sectional shape can be triangular and is adjacent to square-shaped alignment tubes 120. Further, other geometric cross-sectional shapes are possible to form the frame 118.

In a further aspect, the frame 118 can comprise a stability sheet 119. The stability sheet can comprise a piece of material that is coupled to the external surface of the plurality of alignment tubes 120. When the bendable region 116 rotates around the orientation angle θ, tensile (stretching) forces can be exerted on the stability sheet 119. These tensile forces can be oriented parallel to the adjustment member alignment axis AA and a secondary alignment axis AA′. The stability sheet 119 can comprise a resilient material that attempts to resist the tensile forces and return the frame 118 to the prone position. The adjustment strap 108 can be fastened to prevent the stability sheet 119 from pulling the frame 118 back into the prone position.

The stability sheet 119 can be tensile resilient material, such that the material does not tear or unravel after repetitious stretching cycles. For example, the stability sheet 119 can comprise cotton cloth, wool cloth neoprene, knit fabrics, spandex, denim, rubber, latex, burlap, leather, synthetic leather, or polyester. In at least one aspect, the alignment tubes 120 may be fastened to the stability sheet 119 by one or more fasteners or adhesives including but not limited to glue, adhesive, binding agents, chemical binding agents, sewn fabric, stitching, staples, heat compression, and known fastening agents. In at least one aspect, the alignment tubes 120 may be fastened such that each tube is not directly adhered or attached to the other.

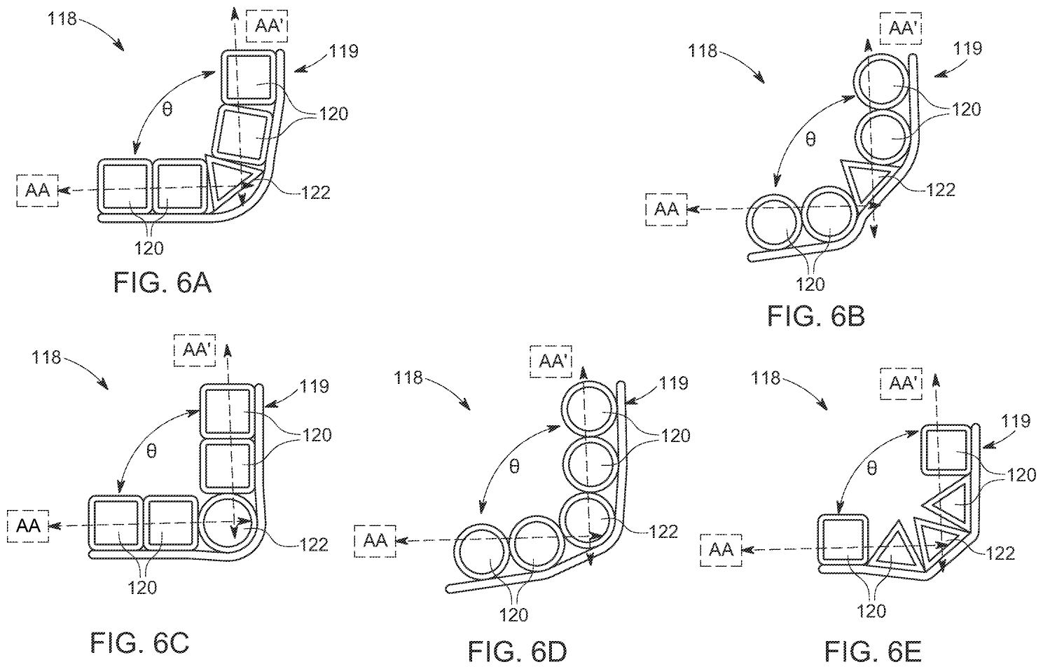

FIGS. 6A, 6B, 6C, 6D and 6E show partial side views of the frame 118 with alternative embodiments of geometric cross-sectional shapes of the alignment tubes 120. As discussed earlier, the stability sheet 119 can be coupled to an exterior surface of the alignment tubes 120. The fulcrum-type alignment tube 122 can be located at the vertex of the orientation angle θ. The variation in the geometric cross-section of adjacent alignment tubes 120 can facilitate the flexion of the frame 118 and define the size of orientation angle θ. Again, the bending fulcrum 122 can be a tube or member that allows adjacent alignment tubes 120 to bend around the orientation angle θ. In one aspect, as shown in FIGS. 6A-6C, the bending fulcrum 122 can be a tube 120 with a cross-sectional shape that differs from the cross-sectional shape of the tube adjacent to the bending fulcrum. In another aspect, the cross-sectional shape of the bending fulcrum 122 can have the same cross-sectional shape when the chosen shape facilitates bending. For example, the cross-sectional shape of a circle or a triangle, as shown in FIGS. 6D and 6E, allows the adjacent tubes to be of the same shape, due to the surfaces of two adjacent tubes not impeding motion relative to each other. As shown in FIGS. 6C and 6D, the curvature of the circular fulcrum 122 facilitates the frame 118 to further decrease the size of the orientation angle. Similarly, for FIG. 6E, the orientation of the triangle's apex for adjacent triangular alignment tubes 120 also facilitates the frame 118 to further decrease the size of the of the orientation angle θ, by allowing the adjacent alignment tubes to generate a curve. A decreased orientation angle θ can allow the user's head to come to rest closer to the user's shoulder, while still providing support. The support for the user's head comes from the stability sheet pulling on the alignment tubes 120 in the attempt to return the frame 118 to the prone position, where the orientation angle is substantially 180 deg. The frame 118 returning to the prone position pushes against the user's head providing the necessary support.

The alignment tubes 120 can be constructed from firm materials with a higher density than the padding 117 or cover material 113 including but not limited to a foam, a rubber, a polyvinyl chloride (PVC), methyl methacrylate (plexiglass), acrylic, plastic, polycarbonate, acetal, fiberglass, acrylonitrile-butadine-styrene (ABS), boltaron thermoplastic, borated polyethylene, polyethylene, nylon, vinyl, polypropylene, polyethylene terephthalate (PET), high-density polyethylene (HDPE), low density polyethylene (LDPE), polymers, and known appropriate rigid and non-rigid materials. In at least one aspect, the material used to form the alignment tubes 120 can be configured to be lightweight and durable.

In one or more aspects, the support apparatus 100 may be placed in one or more configurations that allow a user to ergonomically support the head, neck, spine, joints, muscles, ligaments, and soft tissues while resting, reclining, or sleeping utilizing the support apparatus 102. In one or more aspects, the support apparatus 100 comprising the bendable region 116 of adjustment member 104 can be configured to be fixed at an orientation angle θ further configured to provide additional support to the user's head. In further aspects, the support apparatus 100 may be further adjusted to one or more positions, configurations and orientations using at least one of the adjustment strap 108, the bendable region 116 further comprising a frame 118 wherein frame 118 is configured to provide modular support to the flexion of the bendable region 116. In at least one embodiment, the frame 118 can comprise an arrangement of a plurality of alignment tubes 120 which may be further configured to ergonomically support the head, neck, spine, joints, muscles, ligaments, and soft tissues of a user's head or upper torso while said user is resting, reclining, or sleeping utilizing the support apparatus 102.

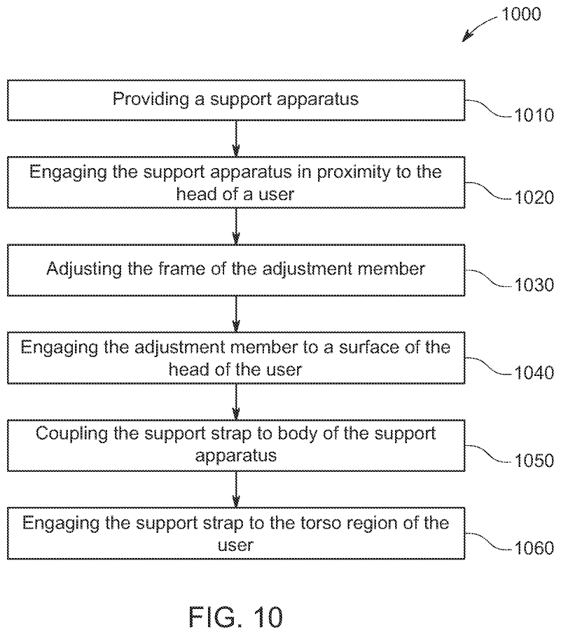

In referring to FIG. 10, a flowchart depicts a method 1000 for using the support apparatus 100. The method 1000 can initiate with Step 1010 which includes providing a support apparatus for the user. Step 1020 can comprise engaging the support apparatus in proximity to the head of a user. In the embodiment depicted in FIG. In Step 1020 the user can place their chin on the chin support member 106. In an alternative embodiment as depicted in FIG. 9, Step 1020 can comprise placing the adjustment member on a shoulder. In Step 1030, the Method 1000 can comprise adjusting the frame of the adjustment member. When adjusting the frame of the adjustment member, the user can then place their head against the adjustment member of the body. In Step 1040, the method comprises engaging the adjustment member to a surface of the head of the user. To adjust their level of comfort, the user can nod their head from side to determine the flexion (size of the orientation angle) of the adjustment member. In a further aspect, the Method 1000 can include stabilizing the orientation angle with an embodiment of the support apparatus comprising an adjustment strap 108. The adjustment strap 108 can be coupled at two sections on adjustment member 116 to restrict the adjustment member from returning to the prone position.

In Step 1050, Method 1000 can include coupling the support strap to body of the support apparatus. The support strap can be coupled to the body by wrapping the support strap over the shoulder on top exterior surface of the adjustment member. In Step 1060, Method 1000 can include engaging the support strap to the torso region of the user. To engage the support strap, the support strap can be traversed across the user's torso and under the opposite arm. The user can then adjust the length of the support strap by coupling fasteners located at the ends and along the length of the support strap. In a further aspect, the method can include securing the apparatus. Securing the support apparatus can include coupling the support strap to the user. In the embodiment as depicted in FIG. 3 and FIG. 9, the support strap further comprises at least one support loop and the at least one support loop engages the user's forearm. In another embodiment, the support strap can be secured by coupling a first portion of the support strap to a second portion of the support strap via a fastener.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way appreciably intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

While the disclosure has been described in connection with what is presently considered to be the most practical and various embodiments, it is to be understood that the disclosure is not to be limited to the disclosed embodiments, but on the contrary, is intended to cover various modifications and equivalent arrangements comprised within the spirit and scope of the appended claims.

What is claimed is:

1. A support apparatus comprising:
body comprising:
an adjustment member including a frame, wherein the frame comprises a bendable region, wherein the bendable region comprises a stability sheet and a plurality of alignment tubes, wherein the stability sheet is coupled to an exterior surface of at least one of the plurality of alignment tubes; and
a support strap detachably coupled to the body.

2. The support apparatus of claim 1, wherein at least two alignment tubes of the plurality of alignment tubes are oriented parallel to each other.

3. The support apparatus of claim 1, wherein the body further comprises a chin support member coupled to the adjustment member.

4. The support apparatus of claim 3, wherein the chin support member and the adjustment member are oriented parallel to each other.

5. The support apparatus of claim 1, wherein the body further comprises an adjustment strap coupled to the adjustment member.

6. The support apparatus of claim 5, wherein the adjustment strap is configured to sustain the bendable region in an angled position.

7. The support apparatus of claim 1, wherein each alignment tube further comprises a tube length axis configured to be substantially perpendicular to an adjustment member axis.

8. The support apparatus of claim 7, a portion of the bendable region is configured to rotate from a prone position to an angled position relative to the adjustment member axis.

9. The support apparatus of claim 1, wherein the body comprises compression-resilient material.

10. The support apparatus of claim 1, wherein the support strap comprises at least one support loop.

11. The support apparatus of claim 1, wherein a portion of the support strap is elastic.

12. A support apparatus comprising:
a body, comprising:
at least one compression-resilient material,
an adjustment strap,
an adjustment member including a frame, wherein the frame comprises a bendable region having:
a stability sheet and,
a plurality of alignment tubes, wherein the stability sheet is coupled to a surface of the plurality alignment tubes and at least two alignment tubes of the plurality of alignment tubes are oriented parallel to each other, and
wherein the adjustment strap is configured to sustain the bendable region in an angled position;
a chin support member coupled to the adjustment member, wherein the chin support member and a portion of the adjustment member are oriented substantially parallel to each other; and
a support strap detachably coupled to the body.

13. The support apparatus of claim 12, wherein the body comprises a wicking material.

14. The support apparatus of claim 12, wherein a portion of the support strap is elastic.

15. The support apparatus of claim 12, wherein the support strap comprises at least one support loop.

16. A method of providing support with a support apparatus, the support apparatus comprising a body comprising: an adjustment member including a frame, wherein the frame comprises a bendable region, wherein the bendable region comprises a stability sheet and a plurality of alignment tubes, wherein the stability sheet is coupled to an exterior surface of at least one of the plurality of alignment tubes and a support strap detachably coupled to the body, that comprises:
engaging the support apparatus in proximity to the head of a user;
adjusting the frame of the adjustment member;
engaging the adjustment member to a surface of the head of the user;
coupling the support strap to body of the support apparatus; and
engaging the support strap to the torso region of the user.

17. The method of claim 16, where in adjusting the frame further comprises, repositioning the spatial orientation of the bendable region between a prone position to an angled position.

18. The method of claim 16 further comprising, wherein the adjustment member further comprises an adjustment strap, coupling the adjustment strap a portion of the body.

19. The method of claim 16, wherein engaging the support strap to the torso of the user comprises wrapping the support strap around the torso of the user.

20. The method of claim 16, further comprising securing the support apparatus, wherein securing the support apparatus comprises at least one of:
coupling the support strap to the user, wherein the support strap further comprises at least one support loop and the at least one support loop engages the user, or
coupling a first portion of the support strap to a second portion of the support strap.
